(19) United States
(12) Patent Application Publication
(10) Pub. No.: US 2022/0203537 A1
(43) Pub. Date: Jun. 30, 2022

(54) CONTROL DEVICE, TASK SYSTEM, CONTROL METHOD AND CONTROL PROGRAM

(71) Applicant: TOYOTA JIDOSHA KABUSHIKI KAISHA, Toyota-shi Aichi-ken (JP)

(72) Inventors: Hirotaka Komura, Setagaya-ku Tokyo (JP); Kunihiro Iwamoto, Nagakute-shi Aichi-ken (JP); Yuta Itozawa, Nagoya-shi Aichi-ken (JP); Yutaro Takagi, Edogawa-ku Tokyo (JP); Yoshiaki Nakamoto, Nisshin-shi Aichi-ken (JP); Junya Ota, Edogawa-ku Tokyo (JP)

(73) Assignee: TOYOTA JIDOSHA KABUSHIKI KAISHA, Toyota-shi Aichi-ken (JP)

(21) Appl. No.: 17/523,270

(22) Filed: Nov. 10, 2021

(30) Foreign Application Priority Data
Dec. 25, 2020 (JP) 2020-216573

(51) Int. Cl. B25J 9/16; B25J 9/00; A61B 5/18; A61B 5/00; A61B 5/0205; A61B 5/11

(52) U.S. Cl. CPC B25J 9/1674; B25J 9/1651; B25J 9/0003; A61B 5/1113; A61B 5/4809; A61B 5/0205; A61B 5/18

(57) ABSTRACT

A control device according to an embodiment of the present disclosure is a control device for a robot that operates in a facility used by a user, and includes a state acquisition unit that acquires a biological state of the user, and a control unit that limits an operation of the robot when the biological state of the user is a sleep onset state. Here, when the biological state of the user is the sleep onset state, the control unit stops the operation of the robot or limits the operation of the robot to a set maximum operation speed or less.

### CROSS-REFERENCE TO RELATED APPLICATION

This application claims priority to Japanese Patent Application No. 2020-216573 filed on Dec. 25, 2020, incorporated herein by reference in its entirety.

### BACKGROUND

### 1. Technical Field

The present disclosure relates to a control device, a task system, a control method and a control program, and for example, a control device, a task system, a control method and a control program for controlling a robot operating in a facility used by a user.

### 2. Description of Related Art

In recent years, robots may operate to execute tasks such as transportation of packages in facilities such as houses. For example, WO 2020/071235 discloses a technique for suppressing a sleeping baby from waking up by operating a robot such that the noise in a target area becomes equal to or lower than a preset noise level.

### SUMMARY

The applicant has found the following issues. The technique disclosed in WO 2020/071235 has an issue that an operation sound of the robot disturbs onset of sleep because the operation of the robot is not restricted when a user is in a sleep onset state.

The present disclosure has been made in view of such issue, and realizes a control device, a task system, a control method, and a control program capable of suppressing disturbance of sleep onset of the user.

A control device according to an aspect of the present disclosure is a control device for a robot that operates in a facility used by a user, and includes: a state acquisition unit that acquires a biological state of the user; and a control unit that limits an operation of the robot when the biological state of the user is a sleep onset state.

In the above-mentioned control device, when the biological state of the user is the sleep onset state, the control unit may stop the operation of the robot.

In the above-mentioned control device, when the biological state of the user is the sleep onset state, the control unit may limit the operation of the robot to a set maximum operation speed or less.

The above-mentioned control device may further include a determination unit that determines whether the user is in the sleep onset state, and when a heart rate or a respiratory rate of the user is less than a set sleep onset threshold, the determination unit may determine that the user is in the sleep onset state.

In the above-mentioned control device, when a plurality of the users is present, the sleep onset threshold may be set corresponding to each of the users.

A task system according to an aspect of the present disclosure includes: the above-mentioned control device; a robot controlled by the control device to execute a task; and a detection unit that is disposed in a room where a user sleeps and detects a biological state of the user.

In the above-mentioned task system, the detection unit may be a biosensor or an imaging sensor, and detect the biological state of the user based on a temporal change in a body movement of the user.

In the above-mentioned task system, when a plurality of the users is present, the detection unit may be disposed in each room where each of the users sleeps, and when at least one of the users is in a sleep onset state, the control device may limit an operation of the robot.

A control method according to an aspect of the present disclosure is a control method for a robot that operates in a facility used by a user, and includes: a step of acquiring a biological state of the user; and a step of limiting an operation of the robot when the biological state of the user is a sleep onset state.

In the above-mentioned control method, when the biological state of the user is the sleep onset state, the operation of the robot may be stopped.

In the above-mentioned control method, when the biological state of the user is the sleep onset state, the operation of the robot may be limited to a set maximum operation speed or less.

The above-mentioned control method may further include a step of determining whether the user is in the sleep onset state, and when a heart rate or a respiratory rate of the user is less than a set sleep onset threshold, the user may be determined to be in the sleep onset state.

In the above-mentioned control method, when a plurality of the users is present, the sleep onset threshold may be set corresponding to each of the users.

A control program according to an aspect of the present disclosure is a control program that controls a robot that operates in a facility used by a user, and that causes a computer to execute: a process of acquiring a biological state of the user, and; a process of limiting an operation of the robot when the biological state of the user is a sleep onset state.

According to the present disclosure, it is possible to realize a control device, a task system, a control method, and a control program capable of suppressing disturbance of sleep onset of the user.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a diagram schematically showing a configuration of a task system according to an embodiment;

FIG. 2 is a perspective view schematically showing a robot according to the embodiment;

FIG. 3 is a side view schematically showing the robot according to the embodiment;

FIG. 4 is a block diagram showing a system configuration of the robot according to the embodiment;

FIG. 5 is a view of a first accommodating portion as viewed from the inside of a house;

FIG. 6 is a perspective view showing a package transported using the robot;

FIG. 7 is a block diagram showing functional elements of a control device according to the embodiment;

FIG. 8 is a flowchart showing a flow of executing a task using the task system according to the embodiment;

FIG. 9 is a diagram showing an example of a hardware configuration included in the control device and the task system; and

FIG. 10 is a diagram schematically showing a configuration of a task system according to another embodiment.

### DETAILED DESCRIPTION OF EMBODIMENTS

Hereinafter, specific embodiments to which the present disclosure is applied will be described in detail with reference to the drawings. However, the present disclosure is not limited to the following embodiments. Further, in order to clarify the explanation, the following description and drawings are simplified as appropriate.

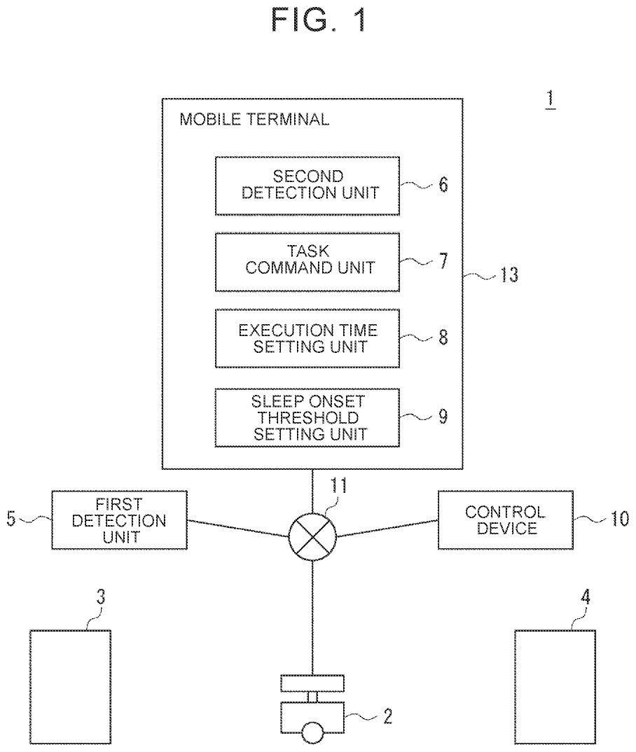

FIG. 1 is a diagram schematically showing a configuration of a task system according to the present embodiment. For example, as shown in FIG. 1, a task system 1 can be used to execute a task in which a robot 2 transports packages stored in a first accommodating portion 3 to a second accommodating portion 4 in a facility used by a user such as a house. Therefore, the number of users is one or more. Here, in the following description, the case of a house as the facility will be described as a representative.

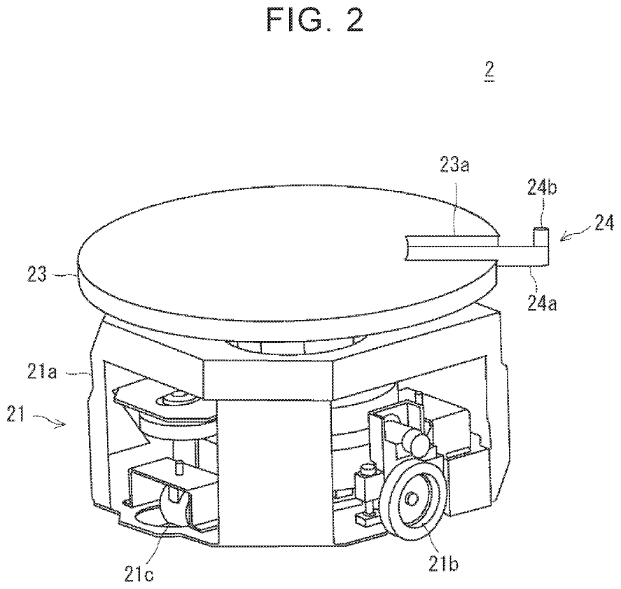

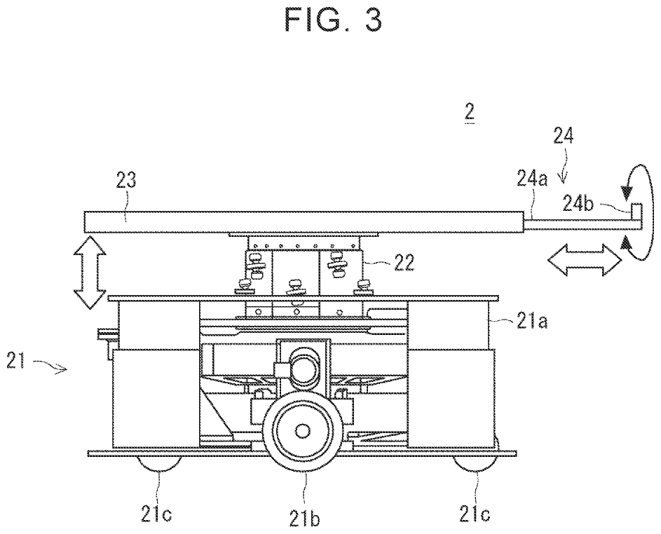

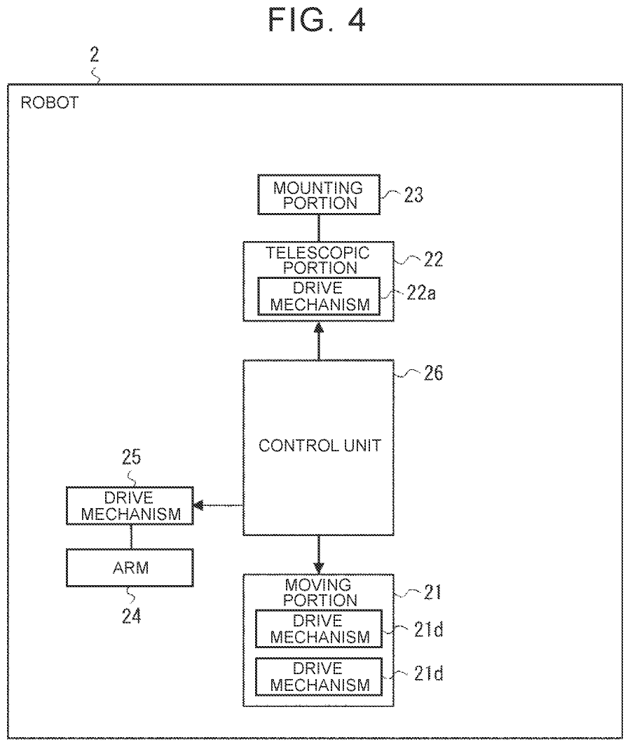

For example, as shown in FIG. 1, the task system 1 includes a first detection unit 5, a second detection unit 6, a task command unit 7, an execution time setting unit 8, and a sleep onset threshold setting unit 9, and a control device 10, in addition to the robot 2. FIG. 2 is a perspective view schematically showing the robot according to the present embodiment. FIG. 3 is a side view schematically showing the robot according to the present embodiment. FIG. 4 is a block diagram showing a system configuration of the robot according to the present embodiment.

The robot 2 is, for example, an autonomous mobile robot and is placed in a house. As shown in FIGS. 1 to 4, the robot 2 includes a moving portion 21, a telescopic portion 22, a mounting portion 23, an arm 24, a drive mechanism 25, and a control unit 26, and is connected to the network 11. Here, the network 11 is, for example, the Internet, and is constructed by a telephone line network, a wireless communication path, Ethernet (registered trademark), or the like.

The moving portion 21 includes a robot body 21a, a pair of right and left drive wheels 21b that is rotatably provided for the robot body 21a, a pair of front and rear driven wheels 21c, and a pair of drive mechanisms 21d. The drive mechanisms 21d rotatably drive the respective drive wheels 21b.

The drive mechanisms 21d each include a motor, a speed reducer, and the like. Each of the drive mechanisms 21d is driven based on control information received from the control unit 26 and rotates the corresponding drive wheel 21b such that the robot body 21a can move forward and rearward, and rotate.

With this configuration, the robot body 21a can move to an arbitrary position. The configuration of the moving portion 21 is an example, and the present disclosure is not limited to this. For example, the number of the drive wheels 21b and the driven wheels 21c of the moving portion 21 may be arbitrary, and a known mechanism can be used as long as the robot 2 can be moved to an arbitrary position.

The telescopic portion 22 is a telescopic mechanism that expands and contracts in an up-down direction. The telescopic portion 22 may be configured as a telescopic type expansion and contraction mechanism. The telescopic portion 22 includes a drive mechanism 22a including a motor, a speed reducer, and the like, and is driven based on the control information received from the control unit 26.

The mounting portion 23 is provided in an upper portion (at a tip) of the telescopic portion 22. The mounting portion 23 moves up and down due to expansion and contraction of the telescopic portion 22. In the present embodiment, the mounting portion 23 is used for loading the package transported by the robot 2.

Then, in order to transport the package, the robot 2 moves together with the package while package is supported by the mounting portion 23. With this configuration, the robot 2 transports the package. However, in the robot 2, when the mounting portion 23 can move up and down, a known mechanism can be used instead of the telescopic portion 22.

The mounting portion 23 includes, for example, a plate material serving as an upper surface and a plate material serving as a lower surface. A space for accommodating the arm 24 and the drive mechanism 25 is provided between the upper surface and the lower surface. In the present embodiment, the shape of the mounting portion 23 is, for example, a flat disk shape, but any other shape may be used.

More specifically, in the present embodiment, the mounting portion 23 is provided with a cutout 23a along a line of flow of the arm 24 such that, when the arm 24 is moved, a protruding portion 24b of the arm 24 does not interfere with the mounting portion 23. The cutout 23a is provided at least on the upper surface of the mounting portion 23.

The mounting portion 23 is provided with the arm 24 that is horizontally moved in and out of the mounting portion 23. The arm 24 includes a shaft portion 24a extending in the horizontal direction and the protruding portion 24b that extends in the direction perpendicular to the shaft portion 24a and is provided at the tip of the shaft portion 24a. That is, in the present embodiment, the arm 24 is L-shaped.

The drive mechanism 25 moves the arm 24 in the horizontal direction (that is, the direction along the shaft portion 24a, in other words, the longitudinal direction of the arm 24) and rotates around the shaft portion 24a, based on the control information received from the control unit 26.

The drive mechanism 25 includes, for example, a motor and a linear guide, and moves the arm 24 in the horizontal direction and rotates the arm 24. As the drive mechanism 25, a known drive mechanism for performing the operations above can be used. The drive mechanism 25 is provided in the mounting portion 23.

As described above, the arm 24 is movable in the horizontal direction, and the protruding portion 24b is rotatable as the arm 24 rotates around the shaft portion 24a. That is, the protruding portion 24b can rotate with the shaft portion 24a as a rotation axis.

The control unit 26 controls the operation of the robot 2. That is, the control unit 26 controls the operations of the moving portion 21, the telescopic portion 22, and the arm 24. The control unit 26 can control the rotation of each drive wheel 21b and move the robot body 21a to an arbitrary position by transmitting the control information to the drive mechanism 21d of the moving portion 21.

Further, the control unit 26 can control the height of the mounting portion 23 by transmitting the control information to the drive mechanism 22a of the telescopic portion 22. Further, the control unit 26 can control the horizontal movement of the arm 24 and the rotation around the shaft portion 24a by transmitting the control information to the drive mechanism 25.

As described above, the control unit 26 may control movement of the robot 2 by executing known control such as feedback control and robust control based on rotation information of the drive wheels 21b detected by a rotation sensor provided for the drive wheels 21b.

Further, the control unit 26 may cause the robot 2 to move autonomously by controlling the moving portion 21 based on information such as distance information detected by a distance sensor such as a camera or an ultrasonic sensor provided for the robot 2 and map information on moving environment.

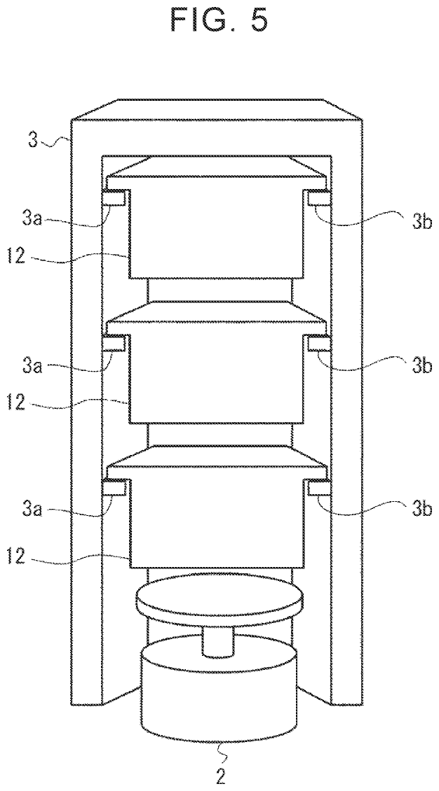

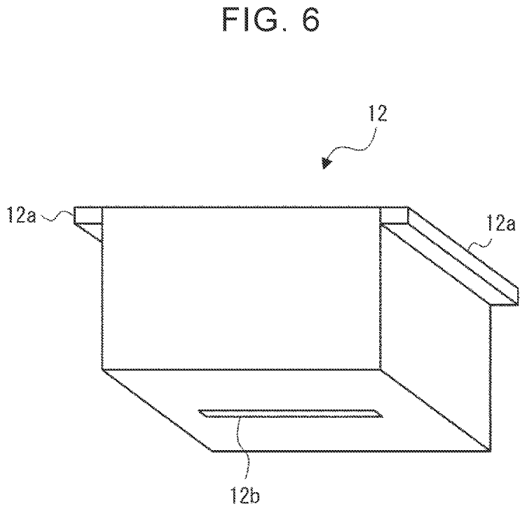

The first accommodating portion 3 stores the packages to be transported by the robot 2. FIG. 5 shows an example of the first accommodating portion, and is a view of the first accommodating portion viewed from the inside of the house. FIG. 6 is a perspective view showing the package transported using the robot. Note that FIG. 5 also shows the robot 2 disposed inside the house with respect to the first accommodating portion 3.

The first accommodating portion 3 is embedded in, for example, a wall of the house and is configured such that the package can be carried in from the outside of the house and can be carried out from the inside of the house by the robot 2. As shown in FIG. 5, the first accommodating portion 3 has a rectangular frame as a basic form, and includes an open portion with which the inside and outside of the house communicate with each other.

In the internal space of the first accommodating portion 3, a plurality of pairs of rails 3a, 3b disposed so as to face each other is provided at intervals in the up-down direction. The rails 3a, 3b extend in a front-rear direction of the first accommodating portion 3. However, the first accommodating portion 3 only needs to have a configuration capable of storing the package 12 such that the package 12 can be carried out at least from the inside of the house.

As shown in FIG. 6, the package 12 is a container having a box shape as a basic form. For example, flanges 12a are provided on respective sides of the package 12. The package 12 is supported in the first accommodating portion 3 as the flanges 12a are supported by the rails 3a, 3b from below.

With this configuration, the package 12 can move in and out of the house in the first accommodating portion 3 along the rails 3a, 3b. Therefore, the package 12 can be carried out from the first accommodating portion 3 by pulling out the package 12 from the inside of the house. However, the package 12 only needs to have a configuration that can be supported by the rails 3a, 3b of the first accommodating portion 3.

As shown in FIG. 6, a groove 12b for hooking the protruding portion 24b of the arm 24 is provided on the bottom surface of the package 12 at a predetermined position. Note that, the package 12 is, for example, a container having a box shape as a basic form. However, the package 12 is not limited to this and may be any object. Any other object can be accommodated in the package 12 as a container.

The second accommodating portion 4 is disposed in the house and stores the package 12 carried by the robot 2. The second accommodating portion 4 only needs to have a configuration capable of storing the package 12, and may have a configuration substantially equal to, for example, the first accommodating portion 3. Note that, a robot (for example, a robot arm) that sorts objects in the package 12 into another package 12 may be provided.

The first detection unit 5 is disposed in each room of the house and detects a biological state of the user present in the house. The first detection unit 5 can be configured by a biosensor such as a radio wave sensor or an ultrasonic sensor, or an imaging sensor such as an infrared camera. When a plurality of the users is present, the first detection unit 5 above is disposed in each room where each user sleeps.

However, the first detection unit 5 only needs to be a sensor capable of detecting the biological state of the user. Further, the first detection unit 5 may include a motion sensor that detects a person, in addition to the sensor that detects the biological state of the person. The first detection unit 5 is connected to the network 11.

The second detection unit 6 detects the position of the user. As shown in FIG. 1, the second detection unit 6 can be configured by, for example, a global positioning system (GPS) receiver mounted on a mobile terminal 13 such as a smartphone owned by the user.

However, the second detection unit 6 can use a known position information system such as another satellite positioning system as long as the system can detect the current position of the user. The second detection unit 6 is connected to the network 11.

The task command unit 7 is operated by the user or another person in order to designate the package 12 for which the task is executed and to input (command) the content (type) of the task to be executed by the robot 2. As shown in FIG. 1, for example, the task command unit 7 is mounted on the mobile terminal 13. The user can make an input by selecting identification information of the package 12 and type information on the task to be executed for the package 12 displayed on a display unit of the mobile terminal 13. The task command unit 7 is connected to the network 11.

The execution time setting unit 8 is operated by the user or another person to set a time slot when the robot 2 is caused to execute a task. As shown in FIG. 1, for example, the execution time setting unit 8 is mounted on the mobile terminal 13. The user can set the time slot when the robot 2 is caused to execute a task via the mobile terminal 13. The execution time setting unit 8 is connected to the network 11.

Here, the “time slot when the robot 2 is caused to execute a task” in the present embodiment is a time slot having a range from the start time of the earliest task to the start time of the latest task desired by the user. That is, the “time slot when the robot 2 is caused to execute a task” in the present embodiment is a task start time slot desired by the user.

However, the “time slot when the robot 2 is caused to execute a task” may be a time slot having a range from the completion time of the earliest task to the completion time of the latest task desired by the user. That is, the “time slot when the robot 2 is caused to execute a task” may be a task completion time slot desired by the user.

Further, the “time slot when the robot 2 is caused to execute a task” may be a time slot having a range from the start time of the earliest task to the completion time of the latest task desired by the user. That is, the “time slot when the robot 2 is caused to execute a task” may be an operation time slot of the robot 2 from the start to the completion of the task desired by the user.

The sleep onset threshold setting unit 9 is operated by the user or another person to set the heart rate or the respiratory rate at which the user enters the sleep onset state. As shown in FIG. 1, for example, the sleep onset threshold setting unit 9 is mounted on the mobile terminal 13. The user can set the heart rate or the respiratory rate at which the user enters the sleep onset state as a sleep onset threshold via the mobile terminal 13. The sleep onset threshold setting unit 9 is connected to the network 11. Here, “sleep onset” indicates a transition from a state of awakening to a state of sleep.

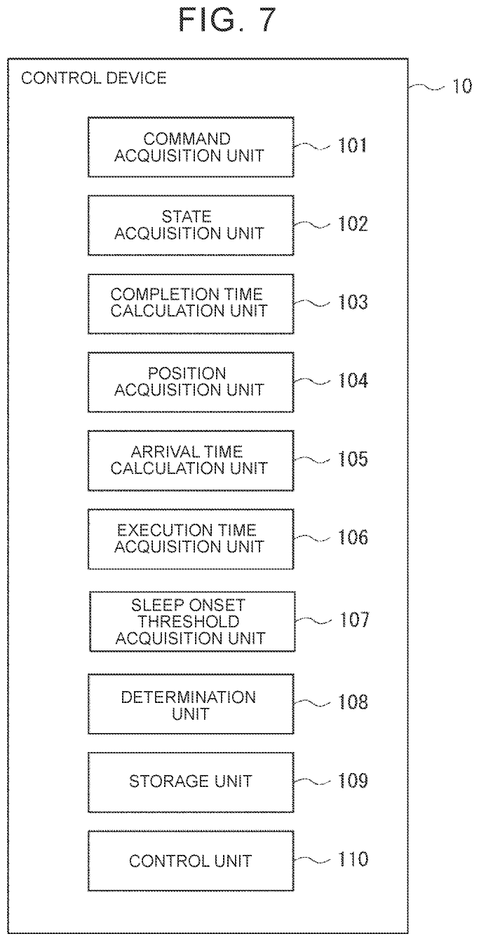

The control device 10 controls the operation of the robot 2 and the like. Here, FIG. 7 is a block diagram showing functional elements of the control device according to the present embodiment. As shown in FIGS. 1 and 7, the control device 10 includes a command acquisition unit 101, a state acquisition unit 102, a completion time calculation unit 103, a position acquisition unit 104, an arrival time calculation unit 105, an execution time acquisition unit 106, and a sleep onset threshold acquisition unit 107, a determination unit 108, a storage unit 109, and a control unit 110. The control device 10 is connected to the network 11.

The command acquisition unit 101 acquires, for example, designation of the package 12 for which a task is executed and the content of the task to be executed by the robot 2. The designation and the content are indicated by the information received from the task command unit 7. Note that, the command acquisition unit 101 may include the task command unit 7. In short, the command acquisition unit 101 only needs to acquire the designation of the package 12 and the content of the task.

Here, the task according to the present embodiment is a task to transport the package 12 in a manner such that the package 12 that is supported by the desired rails 3a, 3b of the first accommodating portion 3 is supported by the desired rails of the second accommodating portion 4.

However, the task is not limited to the task to transport the package 12 from the first accommodating portion 3 to the second accommodating portion 4, and may be a task to move the package 12 such that the package 12 supported by the rails of the first accommodating portion 3 or the rails of the second accommodating portion 4 is supported by other rails.

Further, when the second accommodating portion 4 is provided with the robot arm, the task may be a task to sort objects in the package 12 accommodated in the second accommodating portion 4 into another package 12 by the robot arm without using the robot 2. In short, the content of the task is not limited as long as the task is a task that can be executed using a robot.

The state acquisition unit 102 acquires the biological state of the user. The state acquisition unit 102 acquires the biological state of the user by measuring a temporal change in movement of the body of the user (for example, the heart rate or the respiratory rate) from detection information received from the first detection unit 5.

However, the state acquisition unit 102 may acquire the biological state of the user using a known method. The state acquisition unit 102 may include the first detection unit 5. In short, the state acquisition unit 102 only needs to acquire the biological state of the user.

The completion time calculation unit 103 calculates the time the task is completed. The completion time calculation unit 103 calculates a required time of the task based on the content of the task, a preset movement speed of the robot 2, a telescopic speed of the telescopic portion 22 of the robot 2, an operation speed of the arm 24 of the robot 2, and the like, and calculates the time the task is completed based on the calculated required time of the task and the current time.

The position acquisition unit 104 acquires the position of the user based on the detection information received from the second detection unit 6. At this time, the position acquisition unit 104 may acquire the position of the user in real time. The position acquisition unit 104 may include the second detection unit 6. In short, the position acquisition unit 104 only needs to acquire the position of the user.

The arrival time calculation unit 105 calculates the time the user arrives at the house. The arrival time calculation unit 105 calculates the time the user arrives at the house based on, for example, the current position of the user and a moving speed of the user. At this time, the arrival time calculation unit 105 may update the time the user arrives at the house based on the position information of the user acquired in real time.

However, the arrival time calculation unit 105 may calculate the time the user arrives at the house in the shortest route from the current position of the user using timetable information and required time information of public transportation and the like, and may calculate the time the user arrives at the house using a known calculation method.

The execution time acquisition unit 106 acquires a time slot when the robot 2 is caused to execute a task. The time slot is indicated by the information received from the execution time setting unit 8. The execution time acquisition unit 106 may include the execution time setting unit 8. In short, the execution time acquisition unit 106 only needs to acquire the time slot when the robot 2 is caused to execute a task.

The sleep onset threshold acquisition unit 107 acquires the sleep onset threshold indicated by the information received from the sleep onset threshold setting unit 9. Note that, the sleep onset threshold acquisition unit 107 may include the sleep onset threshold setting unit 9. In short, the sleep onset threshold acquisition unit 107 only needs to acquire the sleep onset threshold.

Although the details will be described later, the determination unit 108 determines whether the user is in the sleep onset state, determines whether the user is present in the house, determines whether the task is completed by the time the user arrives at the house, and determines whether the current time or the like is within the time slot when the robot 2 is caused to execute a task.

The storage unit 109 stores the type information of the task to be executed by the robot 2 or the like, the position information of the first accommodating portion 3, the position information of the rails 3a, 3b of the first accommodating portion 3, the position information of the second accommodating portion 4, the position information of the rails of the second accommodating portion 4, the identification information of the package 12 accommodated in the first accommodating portion 3, the information of the present moving speed of the robot 2, the information of the telescopic speed of the telescopic portion 22 of the robot 2, the information of the operation speed of the arm 24 of the robot 2, the information of the time slot when the robot 2 is caused to execute a task, the sleep onset threshold information, and the like. Here, the identification information of the package 12 and the position information of the rails 3a, 3b in the first accommodating portion 3 are associated with each other.

Although the details will be described later, the control unit 110 controls the robot 2 and the like so as to limit the operation of the robot 2 and the like when the biological state of the user is the sleep onset state. Further, the control unit 110 controls the first detection unit 5 and the second detection unit 6 based on the task command indicated by the information received from the task command unit 7.

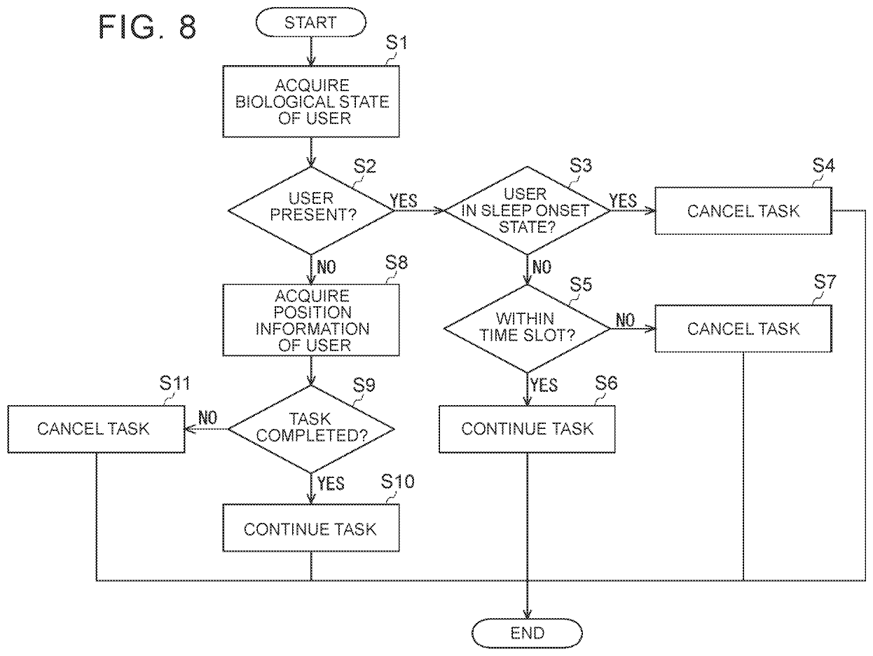

Next, a flow of executing a task using the task system 1 according to the present embodiment will be described. FIG. 8 is a flowchart showing a flow of executing a task using the task system according to the present embodiment.

Here, the following description assumes that a task is executed in which, from the state where the robot 2 is stored in a present storage location in the house, the package 12 accommodated in the first accommodating portion 3 is transported to the second accommodating portion 4 using the robot 2 and the robot 2 returns to the storage location.

Further, it is assumed that the user sets the time slot when the robot 2 is caused to execute the task in advance via the execution time setting unit 8 mounted on the mobile terminal 13, and the information indicating the time slot when the robot 2 is caused to execute the task is stored in the storage unit 109. Further, it is assumed that the user sets the sleep onset threshold in advance via the sleep onset threshold setting unit 9 mounted on the mobile terminal 13, and the information indicating the sleep onset threshold is stored in the storage unit 109.

First, when the user inputs designation of the package 12 and the content of the task via the task command unit 7 mounted on the mobile terminal 13, the task command unit 7 transmits information indicating the designation of the package 12 and the content of the task to the control device 10. With this process, the task system 1 starts the task.

The control unit 110 of the control device 10 controls the first detection unit 5 so as to detect the biological information of the user in the house. When the first detection unit 5 executes detection of the user who is present in the house, the first detection unit 5 transmits the detection information to the control device 10. Then, the state acquisition unit 102 of the control device 10 measures respiration and heartbeat of the user based on the detection information. With this process, the state acquisition unit 102 of the control device 10 acquires the biological state of the user (S1).

Next, the determination unit 108 of the control device 10 determines whether the user is present in the house based on the measurement result of the state acquisition unit 102 (S2). Then, when the determination unit 108 can recognize respiration or heartbeat of the user, the determination unit 108 determines that the user is present in the house (YES in S2). Here, in the case of the plurality of users, for example, when one user is present in the house, the determination unit 108 can determine that the user is present in the house.

Next, when the determination unit 108 of the control device 10 determines that the user is present in the house, the determination unit 108 determines whether the user is in the sleep onset state based on the measurement result of the state acquisition unit 102 (S3). For example, the determination unit 108 determines that the user is in the sleep onset state when the heart rate or the respiratory rate of the user becomes lower than the sleep onset threshold (YES in S3).

Here, when the plurality of users is present, the sleep onset threshold is set for each user. Then, the identification information of each user, the identification information of the room in the house in which each user sleeps, the identification information of the first detection unit 5 disposed for each room where each user sleeps, and the sleep onset threshold set for each user are associated with each other.

With this process, it is possible to accurately determine whether the user is in the sleep onset state. Then, the determination unit 108 of the control device 10 may determine that the user is in the sleep onset state when one of the users is in the sleep onset state. However, the determination unit 108 may determine that the users are in the sleep onset state when the plurality of users is in the sleep onset state. Further, the target user may be set in advance.

When the user is in the sleep onset state, the control device 10 cancels the task (S4). With this process, it is possible to suppress the robot 2 from executing the task while the user is in the sleep onset state, and it is also possible to suppress generation of the operation sound of the robot 2 when the user is in the sleep onset state. Therefore, it is possible to suppress disturbance of sleep onset of the user.

In this case, for example, the control device 10 may control the robot 2 such that the task is executed while the user is not present in the house, or may control the robot 2 to execute the task when the user commands the task again.

On the other hand, for example, when the heart rate or respiratory rate of the user is equal to or higher than the sleep onset threshold, the determination unit 108 of the control device 10 determines that the user is not in the sleep onset state (NO in S3). Then, when the user is not in the sleep onset state, the determination unit 108 of the control device 10 determines whether the current time is within the time slot when the robot 2 is caused to execute the task (S5).

When the current time is within the time slot when the robot 2 is caused to execute the task (YES in S5), the control unit 110 of the control device 10 transmits the control information to the control unit 26 of the robot 2 so as to continue the task.

The control unit 26 of the robot 2 controls the moving portion 21, the telescopic portion 22, and the arm 24 so as to carry out the desired package 12 from the first accommodating portion 3, mount the package 12 on the mounting portion 23, and cause the desired rails of the second accommodating portion 4 to support the package 12 to accommodate the package 12 in the second accommodating portion 4 (S6).

After that, the control unit 26 of the robot 2 controls the moving portion 21 to move the robot 2 to the predetermined storage location, and when the robot 2 arrives at the storage location, the control unit 26 transmits information indicating that the task is completed to the control device 10. When the control device 10 receives the information indicating that the task is completed, the control device 10 ends the task.

When the current time is outside the time slot when the robot 2 is caused to execute the task (NO in S5), the control device 10 cancels the task (S7). With this process, it is possible to suppress the robot 2 from executing the task outside the time slot when the task is executed, and it is also possible to suppress the discomfort of the user when the robot 2 executes the task.

In this case, for example, the control device 10 may control the robot 2 such that the task is executed while the user is not present in the house, or may control the robot 2 to execute the task when the user commands the task again.

When respiration or heartbeat of the user cannot be recognized based on the measurement result of the state acquisition unit 102 of the control device 10, the determination unit 108 of the control device 10 determines that the user is not present in the house (NO in S2). Then, the control unit 110 of the control device 10 controls the second detection unit 6 in order to acquire the detection information of the position of the user.

When the second detection unit 6 acquires the detection information of the position of the user from the GPS satellite, for example, the second detection unit 6 transmits the detection information to the control device 10. With this process, the position acquisition unit 104 of the control device 10 acquires the position of the user (S8).

Next, the completion time calculation unit 103 of the control device 10 calculates the time the task is completed based on the position of the first accommodating portion 3, the positions of the rails 3a, 3b that support the designated package 12 in the first accommodating portion 3, the position of the second accommodating portion 4, the positions of the rails that support the designated package 12 in the second accommodating portion 4, the movement path of the robot 2 in accordance with the task, the preset movement speed of the robot 2, the telescopic speed of the telescopic portion 22 of the robot 2, the operation speed of the arm 24 of the robot 2, and the like. Here, the movement path of the robot 2 in accordance with the task may be calculated by the control unit 26 of the robot 2 and acquired by the completion time calculation unit 103 of the control device 10.

In the present embodiment, the required time for the task can be calculated by adding up the time from when the moving portion 21 of the robot 2 operates to when the moving portion 21 arrives at the first accommodating portion 3 from the predetermined storage location, the time from when the telescopic portion 22 and the arm 24 of the robot 2 operate to when the package 12 is carried out from the first accommodating portion 3 and mounted on the mounting portion 23, the time from when the moving portion 21 of the robot 2 operates to when the moving portion 21 arrives at the second accommodating portion 4, the time from when the telescopic portion 22 and the arm 24 of the robot 2 operate to when the package 12 is carried into the second accommodating portion 4, and the time from when the moving portion 21 of the robot 2 operates to when the robot 2 arrives at the predetermined storage location from the second accommodating portion 4.

Here, when the second accommodating portion 4 includes a robot arm, the time from when the robot arm starts to operate to when sorting of the objects in the package 12 is completed may be added. Then, the time at which the required time of the task calculated from the current time has elapsed can be set as the time the task is completed.

Next, the arrival time calculation unit 105 of the control device 10 calculates the time the user arrives at the house based on the position of the user indicated by the received information. Here, in the case of the plurality of users, the arrival time of the user who arrives at the house earliest can be set as the time the user arrives at the house as a representative. Note that, calculation of the task completion time and calculation of the arrival time of the user may be reversed.

Then, the determination unit 108 of the control device 10 determines whether the task is completed by the time the user arrives at the house (S9). Specifically, the determination unit 108 determines whether the time the task is completed is earlier than the time the user arrives at the house.

When the time the task is completed is earlier than the time the user arrives at the house, the determination unit 108 of the control device 10 determines that the task is completed by the time the user arrives at the house (YES in S9). Then, the control unit 110 of the control device 10 transmits the control information to the control unit 26 of the robot 2 so as to continue the task.

The control unit 26 of the robot 2 controls the moving portion 21, the telescopic portion 22, and the arm 24 so as to carry out the desired package 12 from the first accommodating portion 3, mount the package 12 on the mounting portion 23, and cause the desired rails of the second accommodating portion 4 to support the package 12 to accommodate the package 12 in the second accommodating portion 4 (S10).

After that, the control unit 26 of the robot 2 controls the moving portion 21 to move the robot 2 to the predetermined storage location, and when the robot 2 arrives at the storage location, the control unit 26 transmits information indicating that the task is completed to the control device 10. When the control device 10 receives the information indicating that the task is completed, the control device 10 ends the task. This allows the robot 2 to complete the task by the time the user arrives at the house. Therefore, the user does not feel uncomfortable with the operation sound of the robot 2.

On the other hand, when the time the task is completed is later than the time the user arrives at the house, the determination unit 108 of the control device 10 determines that the task is not completed by the time the user arrives at the house (NO in S9), and cancels the task (S11).

In this case, for example, the control device 10 may control the robot 2 such that the task is executed while the user is not present in the house, or may control the robot 2 to execute the task when the user commands the task again.

As described above, the control device 10, the task system 1, and the control method according to the present embodiment limit the operation of the robot 2 when the user is in the sleep onset state. With this configuration, the volume of the operation sound of the robot 2 when the user is in the sleep onset state can be reduced, thereby suppressing disturbance of sleep onset of the user.

In particular, the operation of the robot 2 is stopped when the user is in the sleep onset state. Therefore, generation of the operation sound of the robot 2 can be suppressed, thereby suppressing disturbance of sleep onset of the user.

Moreover, when the first detection unit 5 is disposed for each room where each user sleeps, it is possible to detect the biological state of each user. Then, when the sleep onset threshold is set for each user, it is possible to accurately determine whether the user is the sleep onset state for each user.

### Other Embodiments

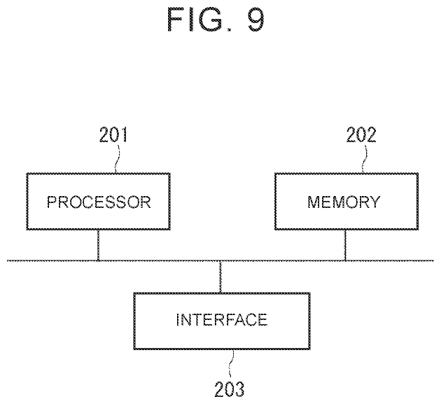

The control device and task system according to the above embodiment may have the following hardware configuration. FIG. 9 is a diagram showing an example of the hardware configuration included in the control device and the task system. As the procedure of processing in the control device and the task system has been described in various embodiments described above, the present disclosure may also take the form of a control method.

The control device shown in FIG. 9 includes a processor 201 and a memory 202 together with an interface 203. A part of the task system 1 and the configuration of the control device 10 described in the above-described embodiment are realized in a manner such that the processor 201 reads and executes a control program stored in the memory 202. That is, the control program is a program for causing the processor 201 to function as a part of the task system 1 or as the configuration of the control device 10. It can be said that the control program is a program for causing the task system 1 and the control device 10 to execute the process in the configuration or a part thereof.

The program described above is stored using various types of non-transitory computer-readable media and can be supplied to a computer (a computer including an information notification device). The non-transitory computer-readable media include various types of tangible storage media. Examples of non-transitory computer-readable media include magnetic recording media (e.g., flexible disks, magnetic tapes, hard disk drives), magneto-optical recording media (e.g., magneto-optical disks). Further, the examples above include a compact disc read-only memory (CD-ROM), a compact disc recordable (CD-R), and a compact disc rewritable (CD-R/W). Further, the examples above include semiconductor memories (e.g., mask ROM, programmable ROM (PROM), erasable programmable ROM (EPROM), flash ROM, random access memory (RAM)). The program may also be supplied to the computer by various types of transitory computer-readable media. Examples of transitory computer-readable media include electrical and optical signals and electromagnetic waves. The transitory temporary computer-readable medium can supply the program to the computer via a wired communication path such as an electric wire and an optical fiber, or a wireless communication path.

The present disclosure is not limited to the above embodiment, and can be appropriately modified without departing from the spirit.

In the above embodiment, when the user is in the sleep onset state, the task of the robot 2 or the like is stopped. However, the task may be executed while the operation of the robot 2 or the like is restricted such that the robot 2 or the like operates at a speed equal to or lower than the set maximum operation speed.

Here, the maximum operating speed of the robot 2 or the like may be any speed at which the robot 2 or the like operates at a speed equal to or lower than the noise level at which the user does not feel uncomfortable with the operation sound of the robot 2 or the like when the robot 2 or the like executes a task while the user is present in the house, and is slower than the normal operation speed of the robot 2. Here, the “normal operation” is an operation in a state where the operation speed is not limited.

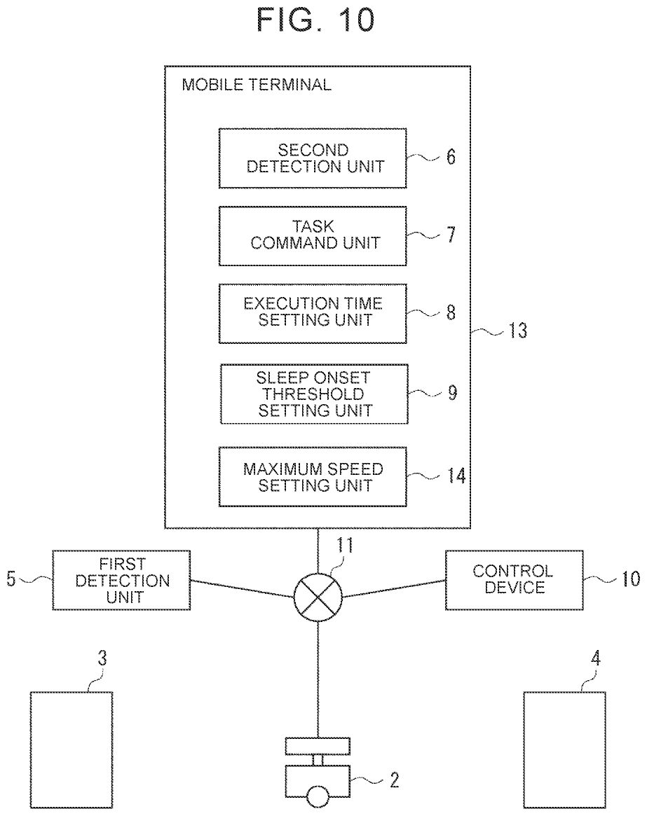

For example, the maximum operation speed of the robot 2 or the like may be set to the operation speed of the robot 2 or the like at which the user does not feel uncomfortable with the operation sound of the robot 2 or the like by actually causing the robot 2 or the like to execute the task, via a maximum speed setting unit 14 mounted on the mobile terminal 13 as shown in FIG. 10.

At this time, the maximum operation speeds of the drive mechanisms 21d, 22a, and 25 of the robot 2 can be selectively set. When the second accommodating portion 4 includes the robot arm, the maximum operation speed of the robot arm may also be included.

Note that, the maximum operation speed of the robot 2 or the like may be set through the supervised learning of a model in which the volume of the operation sound of the robot 2 is an input and the maximum operation speed of the robot 2 or the like is an output, with an evaluation with which a plurality of persons does not feel uncomfortable as a correct answer.

For example, in the above embodiment, when the user is not present in the house, the process in S8 for acquiring the position of the user outside the house and the process in S9 for determining whether the task is completed by the time the user arrives at the house are executed. However, the processes in S8 and S9 may be omitted and the process may proceed to the process in S10. Further, in the above embodiment, when the user is not in the sleep onset state, whether the current time is within the time slot when the robot 2 is caused to execute the task is determined. However, the process in step S5 may be omitted and the process may proceed to the process in S6. In short, when the user is present in the house and the user is in the sleep onset state, the operation of the robot 2 or the like only needs to be restricted.

For example, the configuration of the robot is not limited, and the robot may be any robot as long as the robot can execute a task. The robot may also be a humanoid robot. For example, the robot may be, for example, a housework support robot.

For example, although the robot 2 executes the task in the house in the above embodiment, the same can be executed in a facility such as a hotel where people stay.

What is claimed is:

1. A control device that controls a robot that operates in a facility used by a user, the control device comprising:
a state acquisition unit that acquires a biological state of the user; and
a control unit that limits an operation of the robot when the biological state of the user is a sleep onset state.

2. The control device according to claim 1, wherein when the biological state of the user is the sleep onset state, the control unit stops the operation of the robot.

3. The control device according to claim 1, wherein when the biological state of the user is the sleep onset state, the control unit limits the operation of the robot to a set maximum operation speed or less.

4. The control device according to claim 1, further comprising a determination unit that determines whether the user is in the sleep onset state, wherein when a heart rate or a respiratory rate of the user is less than a set sleep onset threshold, the determination unit determines that the user is in the sleep onset state.

5. The control device according to claim 4, wherein when a plurality of the users is present, the sleep onset threshold is set corresponding to each of the users.

6. A task system, comprising:
the control device according to claim 1;
a robot controlled by the control device to execute a task; and
a detection unit that is disposed in a room where a user sleeps and detects a biological state of the user.

7. The task system according to claim 6, wherein the detection unit is a biosensor or an imaging sensor, and detects the biological state of the user based on a temporal change in a body movement of the user.

8. The task system according to claim 6, wherein:
when a plurality of the users is present, the detection unit is disposed in each room where each of the users sleeps, and
when at least one of the users is in a sleep onset state, the control device limits an operation of the robot.

9. A control method that controls a robot that operates in a facility used by a user, the control method comprising:
a step of acquiring a biological state of the user; and
a step of limiting an operation of the robot when the biological state of the user is a sleep onset state.

10. The control method according to claim 9, wherein when the biological state of the user is the sleep onset state, the operation of the robot is stopped.

11. The control method according to claim 9, wherein when the biological state of the user is the sleep onset state, the operation of the robot is limited to a set maximum operation speed or less.

12. The control method according to claim 9, further comprising a step of determining whether the user is in the sleep onset state, wherein when a heart rate or a respiratory rate of the user is less than a set sleep onset threshold, the user is determined to be in the sleep onset state.

13. The control method according to claim 12, wherein when a plurality of the users is present, the sleep onset threshold is set corresponding to each of the users.

14. A control program that controls a robot that operates in a facility used by a user, and that causes a computer to execute:
a process of acquiring a biological state of the user, and;
a process of limiting an operation of the robot when the biological state of the user is a sleep onset state.
